(19) United States
(12) Patent Application Publication
(10) Pub. No.: US 2026/0229369 A1
(43) Pub. Date: Aug. 6, 2026

(54) SYSTEM AND A DEVICE FOR SPEECH LANGUAGE PATHOLOGY PROGRAMS WITH MEDICAL RECORD ACCESS

(71) Applicant: Jessica M. Wacker, Chicago, IL (US)

(72) Inventor: Jessica M. Wacker, Chicago, IL (US)

(21) Appl. No.: 19/042,873

(22) Filed: Jan. 31, 2025

(51) Int. Cl. G16H 70/00; G16H 10/60; G16H 40/67

(52) U.S. Cl. CPC G16H 70/00; G16H 10/60; G16H 40/67

(57) ABSTRACT

A customized on-line health education platform for providing content to an individual at a remote terminal. The customized on-line health education platform transmits to a remote learning management system, a request for a course associated with a course type. The remote learning management system is operable to provide a plurality of courses based on the request. Information associated with the course is transmitted to or received from the remote learning management system. The information included a delivery method. The course catalog is automatically updated based, at least in part, on the delivery method of the course.

### CROSS-REFERENCES TO RELATED APPLICATIONS

N/A.

### FIELD OF THE TECHNOLOGY

The present disclosure generally relates to HIPPA compliant emergency medical record (EMR) system that also meets all clinical graduate programs and, more specifically, web and mobile platforms for graduate speech-language pathology programs.

### DESCRIPTION OF THE RELATED ART

Several graduate online learning platforms exist for healthcare related course work in general. As such, there are many options for online health education with tailored courses, course management, and online medical records access.

There are many web, mobile, and/or online platforms that provide options for online health education with tailored courses. Different platforms do provide options for what is required of clinical graduate programs like speech-language pathology (SLP) programs to track all the graduation requirements. Nonetheless, these systems are not specific to SLP programs and are not user-friendly, and do not use updated technology resulting in lots of additional work and wasted time for clinical faculty/graduate programs. Additionally, these web, mobile, and/or online platforms are not specialized for speech-language pathology graduate programs.

What is needed is an all-in-one web and/or mobile platform for graduate speech-language pathology programs that is a EMR system that also tracks student background checks, medical records, clinical requirements, supervision hours, competencies, and paperwork during their program required for graduation.

### SUMMARY

In an embodiment, the system includes a customized on-line health education platform for providing content to an individual at a remote terminal. The customized on-line health education platform transmits to a remote learning management system, a request for a course associated with a course type. The remote learning management system is operable to provide a plurality of courses based on the request. Information associated with the course is transmitted to or received from the remote learning management system. The information included a delivery method. The course catalog is automatically updated based, at least in part, on the delivery method of the course.

In another embodiment, the device includes a customized on-line health education platform for providing content to an individual at a remote terminal. The customized on-line health education platform transmits to a remote learning management system, a request for a course associated with a course type. The remote learning management system is operable to provide a plurality of courses based on the request. Information associated with the course is transmitted to or received from the remote learning management system. The information included a delivery method. The course catalog is automatically updated based, at least in part, on the delivery method of the course.

In still another embodiment, a customized on-line health education platform for providing content to an individual at a remote terminal. The customized on-line health education platform transmits to a remote learning management system, a request for a course associated with a course type. The remote learning management system is operable to provide a plurality of courses based on the request. Information associated with the course is transmitted to or received from the remote learning management system. The information included a delivery method. The course catalog is automatically updated based, at least in part, on the delivery method of the course.

### BRIEF DESCRIPTION OF THE DRAWINGS

The present invention is described in detail below with reference to the attached drawing figures, which are incorporated by reference herein and wherein:

FIG. 1 is a block diagram illustrating an exemplary embodiment of the invention;

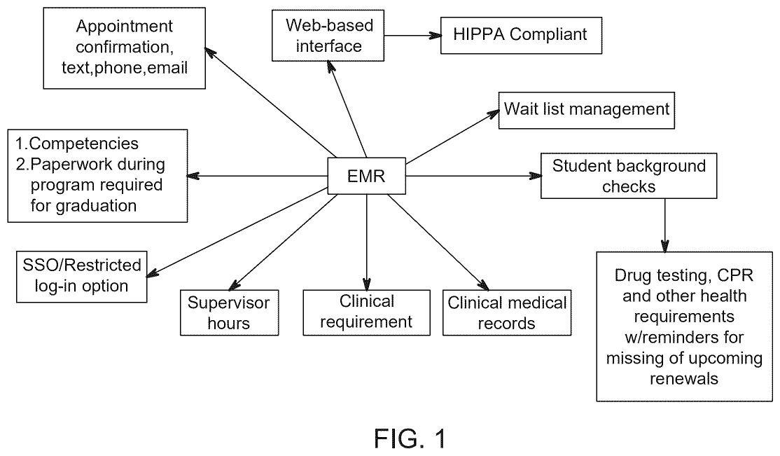

FIG. 2 is a block diagram in accordance with another exemplary embodiment of the invention;

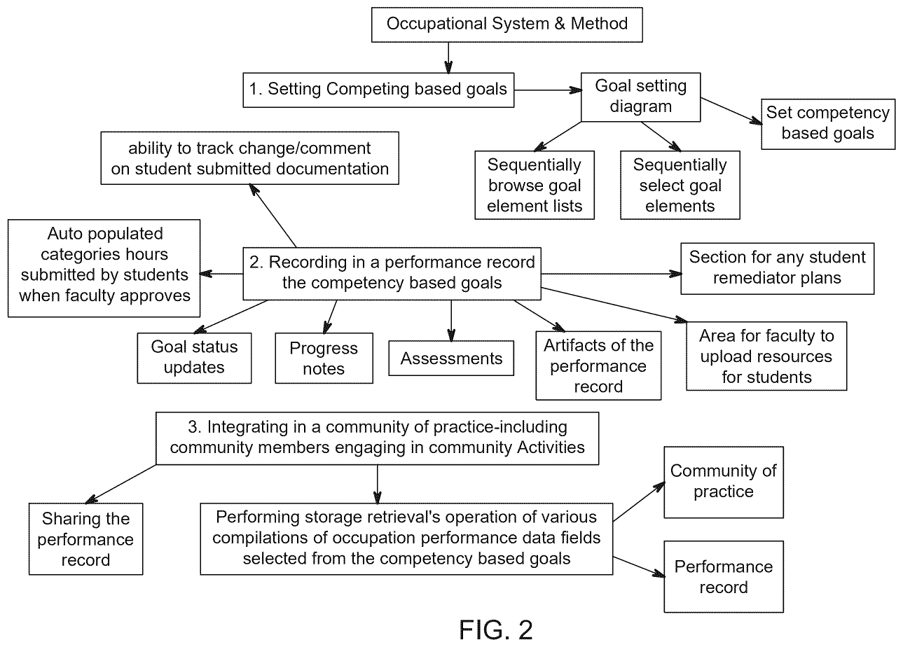

FIG. 3 is a system block diagram with a vehicle in accordance with another exemplary embodiment of the invention; and

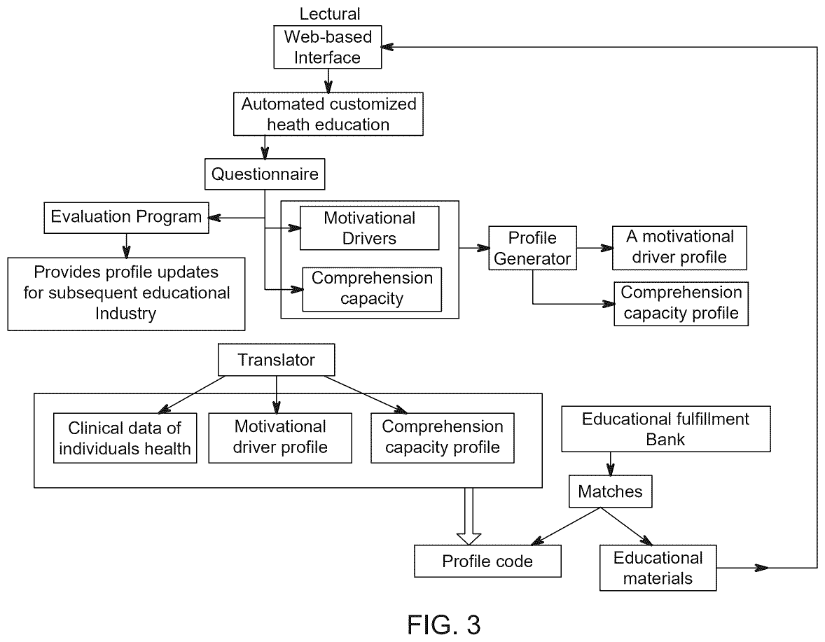

FIG. 4 is a block diagram in accordance with another exemplary embodiment of the invention;

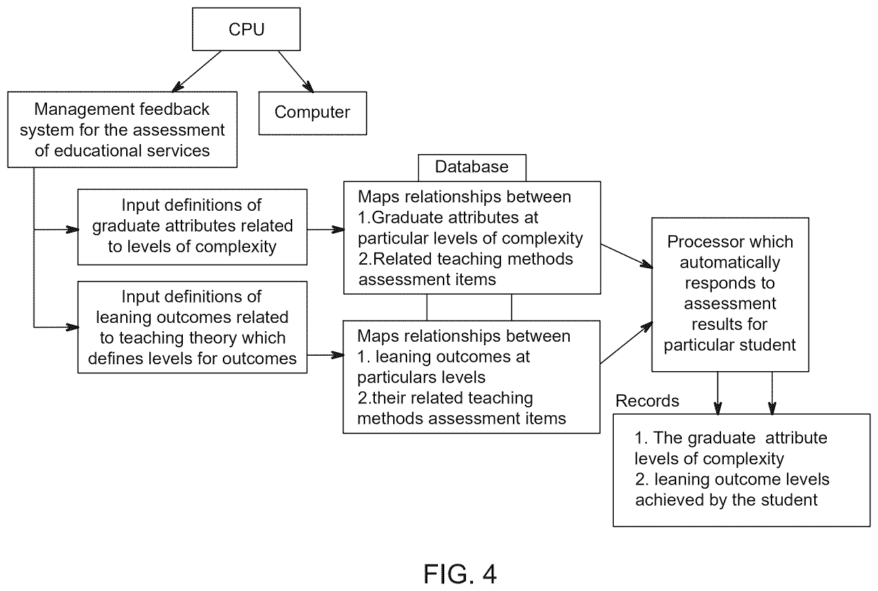

FIG. 5 is a block diagram in accordance with another exemplary embodiment of the invention.

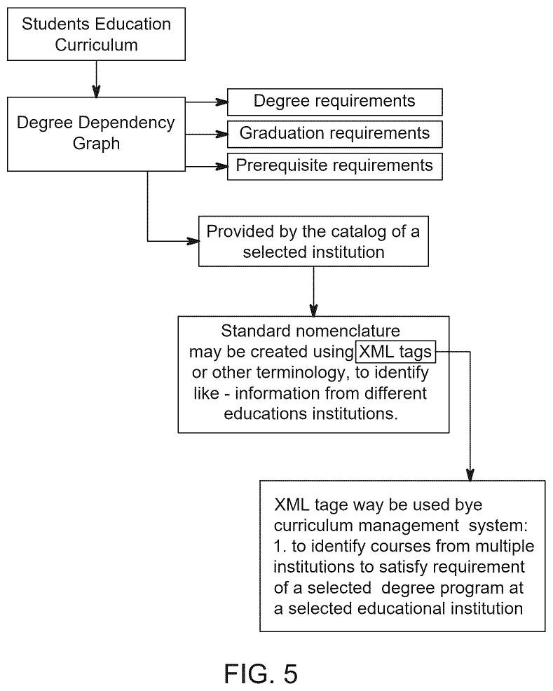

FIG. 6 is a block diagram in accordance with another exemplary embodiment of the invention.

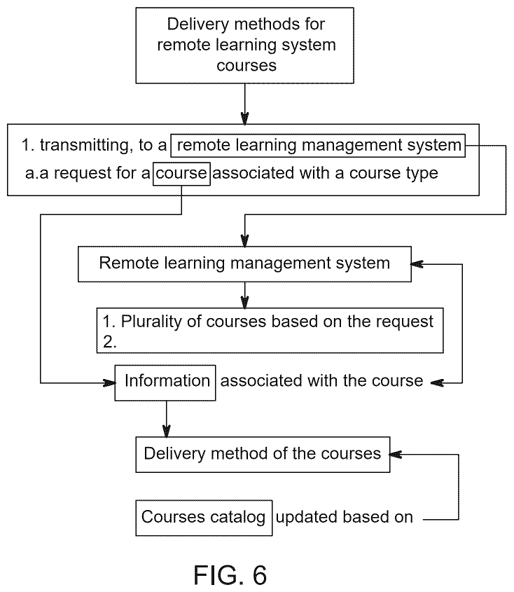

### DETAILED DESCRIPTIONS

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent application, which would still fall within the scope of the claims.

It should also be understood that, unless a term is expressly defined in this patent using the sentence, there is no intent to limit the meaning of that term, either expressly or by implication, beyond its plain or ordinary meaning, and such term should not be interpreted to be limited in scope based on any statement made in any section of this patent application (other than the language of the claims). To the extent that any term recited in the claims at the end of this patent application is referred to in this patent application in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

In an embodiment, the present invention includes web and/or mobile platforms for graduate speech-language pathology programs that are HIPPA compliant, an emergency medical record (EMR) system and which tracks student background checks, medical records, clinical requirements, supervision hours, competencies and paperwork during the program required for graduation. In some embodiments, the present invention includes graduate online learning platforms including online health education with tailored courses, course management, and online medical records access, as well as speech language pathology education and related healthcare fields.

In another embodiment, the present invention includes a system and method, a machine, and an article of manufacture of an occupational performance system and method for achieving occupational performance of competency-based education and work force standards. The occupational performance system and method for achieving occupational performance includes: first, setting competency based goals with a goal setting diagram, to sequentially browse goal element lists, to sequentially select goal elements, to set competency based goals; second, recording in a performance record the competency based goals, goal status updates, progress notes, assessments, and artifacts in the performance record; and third, interacting in a community of practice including community members engaging in community activities, and sharing the performance record, and performing storage, retrieval, and operation of various combinations of occupational performance data fields selected from the competency based goals, performance record, and community of practice to improve occupational performance of the community of practice within the occupational performance system.

In still another embodiment, the present invention includes automated system, and method provides customized health education to an individual at a remote terminal to induce a modification in a health-related behavior of the individual. The automated system includes a questionnaire generator for questioning the individual to determine his or her motivational drivers and comprehension capacity. A profile generator receives answers entered by the individual from the remote terminal and generates a motivational driver profile and a comprehension capacity profile of the individual. A translator receives clinical data relating to a current health condition of the individual and translates the clinical data, the motivational driver profile, and the comprehension capacity profile into a profile code. An educational fulfillment bank matches the profile code to matching educational materials and transfers the matched educational materials to the remote terminal. An evaluation program evaluates educational responses of the individual and provides profile updates for targeting subsequent educational material to the individual based on the educational responses.

In yet another embodiment, the present invention includes a web-based system comprising a server, computer storage and web-portal to provide an integrated management feedback system for the assessment of educational services. The system particularly comprises an interface to input definitions of graduate attributes related to levels of complexity; and input definitions of learning outcomes related to teaching theory which defines levels for outcomes. A database to map relationships between graduate attributes at particular levels of complexity and their related teaching methods and assessment items; and learning outcomes at particular levels and their related teaching methods and assessment items. A processor to automatically respond to the assessment results for a particular student and determine and record both the graduate attribute levels of complexity, and learning outcomes levels, achieved by that student.

In another embodiment, the present invention includes an apparatus, and methods are provided for managing a student's educational curriculum. A degree dependency graph is created for a student corresponding to a degree sought after at a selected educational institution. The degree dependency graph may be created in accordance with degree requirements, graduation requirements, prerequisite requirements, and the like, provided by the catalog of a selected institution. In addition, a standardized nomenclature may be created using XML tags or other terminology, to identify like information from different educational institutions. The XML tags may be used by a curriculum management system to identify courses from multiple institutions to satisfy requirements of a selected degree program at a selected institution.

In still another embodiment, the present invention includes delivery methods for remote learning system courses. In one embodiment, a method includes transmitting, to a remote learning management system, a request for a course associated with a course type. The remote learning management system is operable to provide a plurality of courses based on the request. Information associated with the course is transmitted to or received from the remote learning management system. The information included a delivery method. The course catalog is automatically updated based, at least in part, on the delivery method of the course.

Many different arrangements of the various components depicted, as well as components not shown, are possible without departing from the spirit and scope of the present invention. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Not all steps listed in the various figures need be carried out in the specific order described.

What is claimed is:

1. A system having a customized on-line health education platform for providing content to an individual at a remote terminal comprising:
a customized on-line health education platform that transmits information to a remote learning management system, wherein the information includes a request for a course associated with a course type; wherein the remote learning management system is operable to provide a plurality of courses based on the request; and a course catalog that is automatically updated based, at least in part, on a delivery method of the course.

2. The system of claim 1, wherein the information includes a delivery method.

3. The system of claim 1, wherein the customized on-line health education platform is a web platform.

4. The system of claim 1, wherein the customized on-line health education platform is a mobile platform.

5. The system of claim 1, wherein the customized on-line health education platform is HIPPA compliant.

6. The system of claim 1, wherein the customized on-line health education platform includes a EMR system.

7. The system of claim 1, wherein the customized on-line health education platform includes tracking student background checks.

8. A customized on-line health education platform for providing content to an individual at a remote terminal comprising:
a customized on-line health education platform that transmits information to a remote learning management system, wherein the information includes a request for a course associated with a course type; wherein the remote learning management system is operable to provide a plurality of courses based on the request; and a course catalog that is automatically updated based, at least in part, on a delivery method of the course.

9. The device of claim 8, wherein the information includes a delivery method.

10. The device of claim 8, wherein the customized on-line health education platform is a web platform.

11. The device of claim 8, wherein the customized on-line health education platform is a mobile platform.

12. The device of claim 8, wherein the customized on-line health education platform is HIPPA compliant.

13. The device of claim 8, wherein the customized on-line health education platform includes a EMR system.

14. The device of claim 8, wherein the customized on-line health education platform includes tracking student background checks.
